(19) United States
(12) Patent Application Publication
(10) Pub. No.: US 2009/0060963 A1
(43) Pub. Date: Mar. 5, 2009

(54) Wearing Article Capable of Promoting Human Health

(75) Inventor: Chin-Hsing Hsieh, Yongkang City (TW)

Correspondence Address: Brinks Hofer Gilson & Lione/Ann Arbor, 524 South Main Street, Suite 200, Ann Arbor, MI, 48104, US

(73) Assignee: Homeway Technology Co., Ltd., Yongkang City (TW)

(21) Appl. No.: 12/190,327

(22) Filed: Aug. 12, 2008

(30) Foreign Application Priority Data
Aug. 28, 2007 (TW) 096214304

(51) Int. Cl. A61L 15/18; A61L 15/00; A01N 25/34

(52) U.S. Cl. 424/404; 424/618; 607/100; 424/403

(57) ABSTRACT

A wearing article includes an article element and a fastening mechanism. The article element includes at least one pack unit that has a fabric housing and a flexible pad body fitted into the fabric housing and including a thin elastic matrix of an elastic material and a plurality of functional particles dispersed in the matrix and capable of providing at least one of the functions consisting of emitting far-infrared rays, emitting negative ions, and destroying bacteria. The fastening mechanism is secured to the pack unit so as to permit wearing of the pack unit by the human body and abutment of the fabric housing against the human body.

### CROSS-REFERENCE TO RELATED APPLICATION

This application claims priority of Taiwanese Application No. 096214304, filed on Aug. 28, 2007.

### BACKGROUND OF THE INVENTION

1. Field of the Invention

This invention relates to a wearing article, more particularly to a wearing article including a flexible pad body containing functional particles capable of emitting far-infrared rays and negative ions and destroying bacteria.

2. Description of the Related Art

Some natural minerals, such as silicate minerals, have been found to have functions of emitting far-infrared rays and/or negative ions which can improve blood circulation and stimulate the body's natural healing processes. In addition, silver particles have been used in destroying bacteria in some applications. However, there is a lack of applications on how the natural minerals and/or the silver particles can be worn on the human body and how the effectiveness of the natural minerals on blood circulation, stress relief, recovery from fatigue, etc., can be enhanced.

### SUMMARY OF THE INVENTION

Therefore, the object of the present invention is to provide a wearing article containing functional particles dispersed in a thin elastic matrix that can be conformed to the user's body so as to be effective in providing the functions of emitting far-infrared rays and/or negative ions and destroying bacteria.

According to this invention, there is provided a wearing article that comprises an article element and a fastening mechanism. The article element includes at least one pack unit having a fabric housing and a flexible pad body fitted into the fabric housing and including a thin elastic matrix of an elastic material and a plurality of functional particles dispersed in the matrix and capable of providing at least one of the functions consisting of emitting far-infrared rays, emitting negative ions, and destroying bacteria. The fastening mechanism is secured to the pack unit so as to permit wearing of the pack unit by the human body and abutment of the fabric housing against the human body.

### BRIEF DESCRIPTION OF THE DRAWINGS

Other features and advantages of the present invention will become apparent in the following detailed description of the preferred embodiments of this invention, with reference to the accompanying drawing, in which:

FIG. 1 is a perspective view of the first preferred embodiment of a wearing article according to this invention;

FIG. 2 is a perspective view of a flexible pad body of the first preferred embodiment;

FIG. 3 is a fragmentary sectional view of the first preferred embodiment;

FIG. 4 is a perspective view of the second preferred embodiment of the wearing article according to this invention;

FIG. 5 is an exploded perspective view of the third preferred embodiment of the wearing article according to this invention; and

FIG. 6 is a sectional view of the third preferred embodiment.

### DETAILED DESCRIPTION OF THE PREFERRED EMBODIMENTS

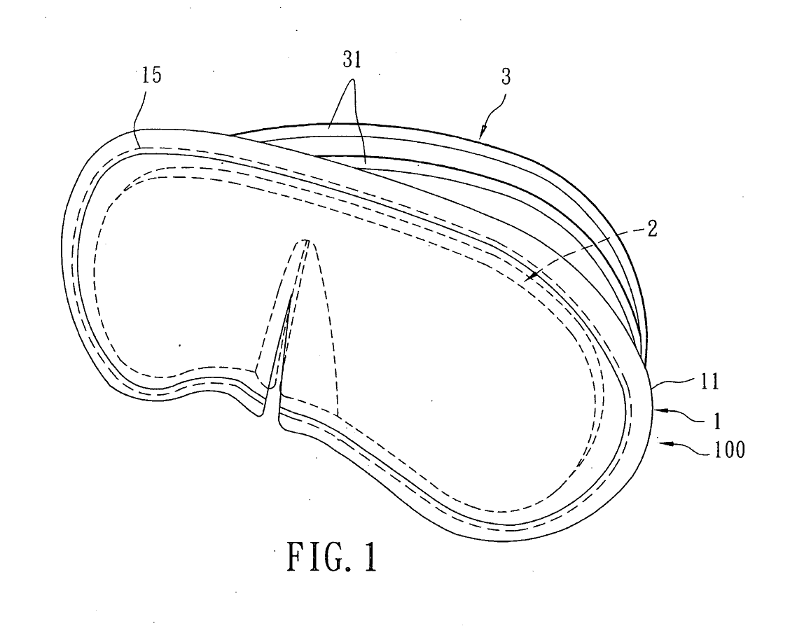

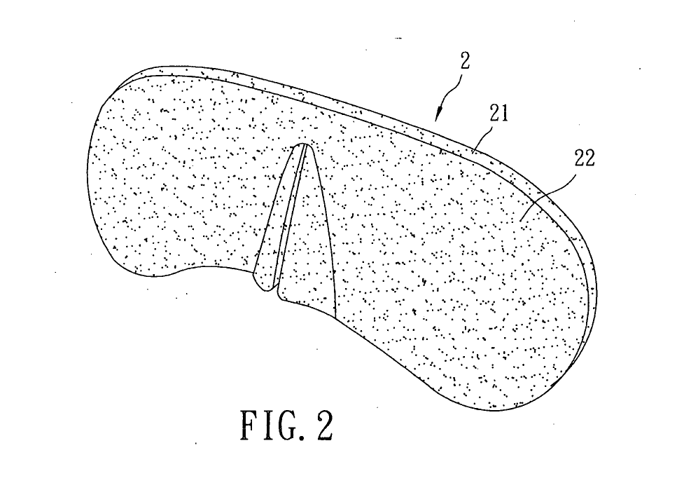

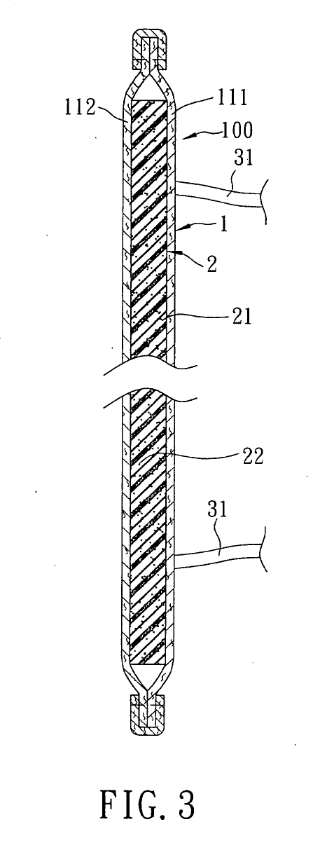

FIGS. 1 to 3 illustrate the first preferred embodiment of a wearing article according to this invention. The wearing article includes an article element and a fastening mechanism 3. The article element includes at least one pack unit 100 having a fabric housing 1 and a flexible pad body 2 fitted into the fabric housing 1 and including a thin elastic matrix 21 of an elastic material and a plurality of functional particles 22 dispersed in the matrix 21 and capable of providing at least one of the functions consisting of emitting far-infrared rays, emitting negative ions, and destroying bacteria. The fastening mechanism 3 is secured to the pack unit 100 so as to permit wearing of the pack unit 100 by the human body and abutment of the fabric housing 1 against the human body.

In this embodiment, the fabric housing 1 includes first and second fabric sheets 111, 112 which are stitched through a thread line 15 along peripheries thereof so as to enclose the flexible pad body 2.

The elastic material can be thermoplastic elastomers or thermoplastic rubbers, such as SIS, SBS, and SEBS. The elastic matrix 21 preferably has a thickness ranging from 1 to 2 mm.

Preferably, the functional particles 22 are made from silicate minerals capable of emitting far-infrared rays and/or negative ions. Preferably, the functional particles 22 contain silicate minerals and silver particles.

In this embodiment, the pad body 2 has a structure conforming to a face area that covers eyes, nose, and temples so as to provide the function(s) of the functional particles 22 to the covered area of the user's face.

In this embodiment, the fastening mechanism 3 includes a pair of elastic strips 31 connected to two opposite ends of the fabric housing 1.

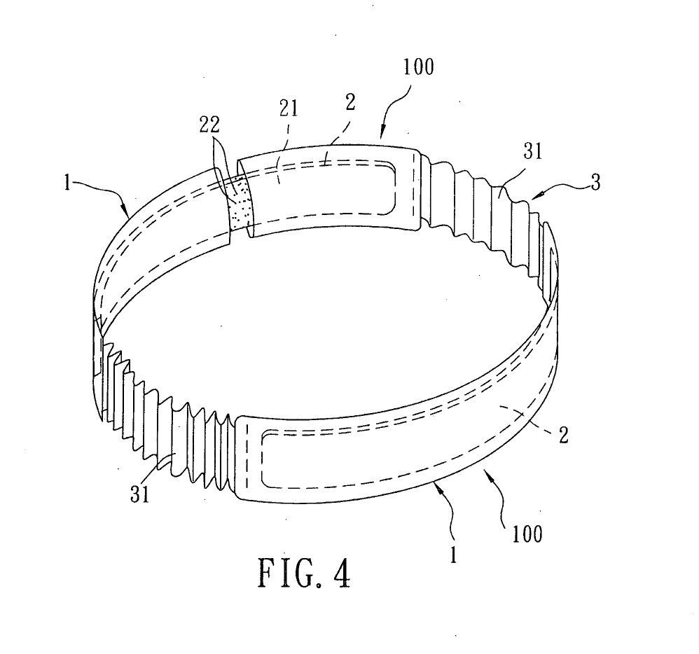

FIG. 4 illustrates the second preferred embodiment of the wearing article according to this invention. The second preferred embodiment differs from the previous embodiment in that the article element is in the form of a belt, and includes a pair of the pack units 100, and that the fastening mechanism 3 includes a pair of pleated bands 31, each of which interconnects two adjacent ends of the pack units 100 through stitching. The wearing article of the second preferred embodiment can be worn on the user's head, neck, wrist, elbow, leg, knee, thigh, waist, etc.

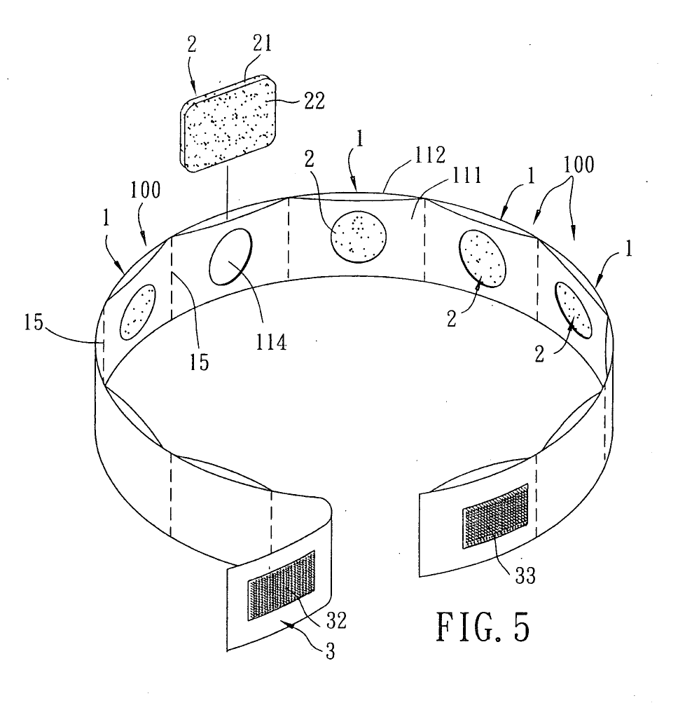

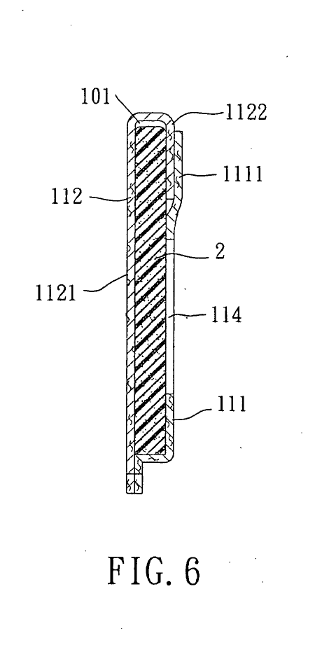

FIGS. 5 and 6 illustrate the third preferred embodiment of the wearing article according to this invention. The third preferred embodiment differs from the previous embodiment in that the article element is in the form of a belt, includes a plurality of the pack units 100 that are interconnected through stitching lines 15, and has two opposite ends that are provided with first and second interlocking members 32, 33 which are releasably engageable with each other in a hook-and-loop manner. In this embodiment, the first and second interlocking members 32, 33 are made from Velco® means.

In this embodiment, the first fabric sheet 111 has an end portion 1111 and the second fabric sheet 112 has a main portion 1121 and a folded portion 1122 folded from one end of the main portion 1121. The end portion 1111 of the first fabric sheet 111 cooperates with the main portion 1121 of the second fabric sheet 112 to define an opening 101 therebetween. The folded portion 1122 of the second fabric sheet 112 is movable relative to the first fabric sheet 111 and the main portion 1121 of the second fabric sheet 112 so as to cover and uncover the opening 101. The folded portion 1122 of the second fabric sheet 112 is attached to the end portion 1111 of the first fabric sheet 111. Moreover, the first fabric sheet 111 of the fabric housing 1 of each of the pack units 100 is formed with an aperture 114 for exposing a portion of the flexible pad body 2 disposed therein. The third preferred embodiment permits removal of the pad bodies 2 from the wearing article so as to permit cleaning of the interconnected fabric housings 1 of the pack units 100.

With the inclusion of the flexible pad body 2 and the fastening mechanism 3 in the wearing article of this invention, applications of the natural minerals, which are capable of emitting far-infrared rays and/or the negative ions, can be broadened and effectiveness of the natural minerals in their function(s) can be enhanced.

While the present invention has been described in connection with what are considered the most practical and preferred embodiments, it is understood that this invention is not limited to the disclosed embodiments but is intended to cover various arrangements included within the spirit and scope of the broadest interpretation so as to encompass all such modifications and equivalent arrangements.

What is claimed:

1. A wearing article capable of promoting human health, characterized by:
an article element and a fastening mechanism, said article element including at least one pack unit that has a fabric housing and a flexible pad body fitted into said fabric housing and including a thin elastic matrix of an elastic material and a plurality of functional particles dispersed in said elastic matrix and capable of providing at least one of the functions consisting of emitting far-infrared rays, emitting negative ions, and destroying bacteria, said fastening mechanism being secured to said pack unit so as to permit wearing of said pack unit by the human body and abutment of said fabric housing against the human body.

2. The wearing article of claim 1, characterized in that said functional particles are made from silicate minerals capable of emitting far-infrared rays.

3. The wearing article of claim 1, characterized in that said functional particles are made from a material capable of emitting negative ions.

4. The wearing article of claim 1, characterized in that said functional particles are made from silver particles capable of destroying bacteria.

5. The wearing article of claim 1, characterized in that said fabric housing includes first and second fabric sheets which are stitched along peripheries thereof so as to enclose said flexible pad body.

6. The wearing article of claim 1, characterized in that said fastening mechanism includes a pair of elastic strips connected to two opposite ends of said fabric housing.

7. The wearing article of claim 1, characterized in that said article element is in the form of a belt, and includes a pair of said pack units, said fastening mechanism including a pair of pleated bands, each of which interconnects two adjacent ends of said pack units.

8. The wearing article of claim 1, characterized in that said article element is in the form of a belt, includes a plurality of said pack units that are interconnected, and has two opposite ends that are provided with first and second interlocking members which are releasably engageable with each other in a hook-and-loop manner.

9. The wearing article of claim 8, characterized in that said fabric housing of each of said pack units includes first and second fabric sheets that are connected to each other to enclose said flexible pad body, said first fabric sheet having an end portion, said second fabric sheet having a main portion and a folded portion folded from one end of said main portion, said end portion of said first fabric sheet cooperating with said main portion of said second fabric sheet to define an opening therebetween, said folded portion of said second fabric sheet being movable relative to said first fabric sheet and said main portion of said second fabric sheet so as to cover and uncover said opening, said folded portion of said second fabric sheet being attached to said end portion of said first fabric sheet.

10. The wearing article of claim 9, characterized in that said first fabric sheet of said fabric housing of each of said pack units is formed with an aperture for exposing a portion of said flexible pad body disposed therein.
